(19) United States
(12) Patent Application Publication
(10) Pub. No.: US 2002/0127286 A1
(43) Pub. Date: Sep. 12, 2002

(54) Method for the treatment of human or animal cells, tissue cultures and/or organs outside the human or animal body

(76) Inventors: Mathis-Heinrich Kreuter, Walenstadt (CH); Rene Flachsmann, Richterswil (CH)

Correspondence Address: William L. Mathis, BURNS, DOANE, SWECKER & MATHIS, L.L.P., P.O. Box 1404, Alexandria, VA, 22313-1404, US

(21) Appl. No.: 10/024,702

(22) Filed: Dec. 21, 2001

Related U.S. Application Data
(62) Division of application No. 08/957,367, filed on Oct. 24, 1997.
(63) Continuation-in-part of application No. 08/957,367, filed on Oct. 24, 1997.

(30) Foreign Application Priority Data
Oct. 24, 1996 (CH) 1996 2606/96

(51) Int. Cl. A61K 35/78; A01N 1/02

(52) U.S. Cl. 424/729; 435/1.1

(57) ABSTRACT

The present invention is directed to a method for the treatment of human or animal cells, tissue cultures and/or organs outside the human or animal body, wherein said human or animal cells, tissues and/or organs are in need of:

(i) prevention or considerable reduction of the formation of tissue necrosis and/or atrophy and/or the prevention or considerable reduction of the premature mortification of vascularized and non-vascularized cells and cellular tissues/colonies, and

(ii) prevention or considerable reduction of adhesion of a human or animal cell to cells or cell unions of different non-histocompatible tissue types while promoting adhesion of said cell to cells or cell unions of the same tissue type;

comprising administering to said human or animal cells, tissue cultures and/or organs outside the human or animal body and in need of treatment a medicament, a medical care product, a food product or an ingredient for tissue culture media, which comprises a treatment-effective amount of a partial extract of non-fermented Camellia sinensis L., obtained by extracting non-fermented Camellia sinensis L. with a mixture of alcohol and water, wherein said extract comprises phenylchroman derivatives, caffeine, theobromine, glutaminic acid-ethylamide, flavonoides and plant acids.

The present invention is directed to a method for the treatment of human or animal cells, tissue cultures and/or organs outside the human or animal body.

Preparations of Camellia sinensis L. for medical and cosmetic applications are known; see “Hagers Handbuch der Pharmazeutischen Praxis”, Vol. 4, Drogen A-D, Springer-Verlag, (1992), pages 628 - 640.

Due to its content compounds Camellia sinensis L. has a central stimulating, moderate diuretic, in dependence of the extraction time more or less strong constipatory/anti-diarrhoeal, the heart activity promoting and possibly antiarteriosclerotic effect, see Stagg G. V., Millin D. J., (1975), J. Sci. Food. Agric. 26, pages 1439-1459.

The further prior art concerning Camellia sinensis L. is described in Zeitschrift fur Phytotherapie 17, (1995), pages 231-250.

So are especially assigned to the in Camellia sinensis L. contained polyphenols an antioxidant efficacy, which shall protect the human body from so called radicals.

The polyphenols are also assigned to have an anti-inflammatory efficacy.

An inhibition of the tumor formation by means of Camellia sinensis L. extracts has been proved in animal experiments and is supported by epidemiological-studies.

Also mentioned are virostatic and bacteriostatic effects of Camellia sinensis L. extracts.

In JP 07/101 837 is described an agent having anti-dandruff activity. As active component this agent contains an alcoholic extract of not further specified tea leaves.

In JP 06/056 687 is described an agent with which is removed dental tartar, and with which the formation of dental tartar is prevented.

As active components this agent contains an aqueous or a with a hydrophilic organic solvent obtained extract of tea leaves (Camellia sinensis).

For the claimed activity has been made responsible the strong antimicrobial effect against periodontotic pathogenic bacterias.

In JP 08/073 350 is described an agent for the improvement of cerebral functions.

As active component is mentioned theanine (glutamic acid ethylamide). This component may be contained in tea extracts.

In JP 06/100 442 is described an anti-stress agent for the prevention or mitigation of mental and physical diseases due to stress.

As active component this agent contains L-theanine of native origin as such or in the form of a pharmaceutically acceptable salt, for example the hydrochloride.

In U.S. Pat. No. 5,071,653 are described processes for the preparation of extracts of Camellia sinensis.

These extracts promote the growth of bifido-bacterias.

These extracts contain in contrary to tea infusions, partial and complete extracts of not fermented Camellia sinensis neither flavone glucosides nor polyphenols, and also no therpenoides and no lipophilic compounds.

With these in U.S. Pat. No. 5,071,653 described processes are made highly selective fractionations of Camellia sinensis content compounds, which have a completely unusual activity spectra, that is the promotion of the growth of intestine bacterias.

Quite surprisingly were found new use possibilities of partial or complete extracts of not fermented Camelia sinensis L.

The present invention is directed to a method for the treatment of human or animal cells, tissue cultures and/or organs outside the human or animal body, wherein said human or animal cells, tissues and/or organs are in need of:

(i) prevention or considerable reduction of the formation of tissue necrosis and/or atrophy and/or the prevention or considerable reduction of the premature mortification of vascularized and non-vascularized cells and cellular tissues/colonies, and

(ii) prevention or considerable reduction of adhesion of a human or animal cell to cells or cell unions of different non-histocompatible tissue types while promoting adhesion of said cell to cells or cell unions of the same tissue type;

comprising administering to said human or animal cells, tissue cultures and/or organs outside the human or animal body and in need of treatment a medicament, a medical care product, a food product or an ingredient for tissue culture media, which comprises a treatment-effective amount of a partial extract of non-fermented Camellia sinensis L., obtained by extracting non-fermented Camellia sinensis L. with a mixture of alcohol and water, wherein said extract comprises phenylchroman derivatives, caffeine, theobromine, glutaminic acid-ethylamide, flavonoides and plant acids.

A preferred embodiment is the cultivation and propagation and/or the three-dimensional reconstruction of cartilage cells, gingiva cells, hair root cells, skin cells and skin tissues, retina cells and retina tissue, heart muscle cells and heart muscle tissues, or liver cells and liver tissues.

The extract of Camellia sinensis L. used for the examinations described below was prepared as follows.

6 kg dried, not fermented Camellia sinensis L. (folia) were extracted under stirring with 60 kg of a mixture of 8 parts by weight of ethanol and 2 parts by weight of water at a temperature from 25° C. to 35° C. during 2 hours.

Then was filtered, and the solvent mixture was evaporated at a pressure from 50 mbar to 150 mbar and a temperature from 30° C. to 40° C. up to a complete evaporation of ethanol.

From the so obtained concentrate were removed according to EP 0 730 830 A1 the undesired lipophilic contaminations and residues.

The so purified extract was then subjected to a process for a decrease of the bacterial count (30 seconds at a temperature of 120° C.).

Then this extract was spray dried.

There was obtained 1 kg of native dry extract having the following analysis datas.

50% m/m phenylchroman derivatives, calculated as epicatechin (HPLC),

6% m/m caffeine (HPLC)

1% m/m theobromine (HPLC).

In addition were detected qualitatively:

glutaminic acid-ethylamide

flavonoides and

plant acids (HPTLC).

This product is obtainable from the company Emil Flachsmann AG in CH-8820 Wädenswil/Switzerland under the denotation “EFLA 85942”.

The following examples illustrate the present invention.

### EXAMPLE 1

The product “EFLA 85942” was tested on its effects on a special cell model, that is the multicellular spheroids.

These spheroids are ball-shaped cell aggregates, which contain in suspension culture at a diameter of about 1 mm up to 100'000 cells, and as a rule reflect better the biological relations of cell unions in vivo than conventional monolayer cultures.

Tested was the influence of the product “EFLA 85942” onto the volume growth of these spheroids as a function of the cultivation length and at different concentrations of “EFLA 85942” in comparison to control cultures.

The stock solution was prepared as follows:

“EFLA 85942” was dissolved at a concentration of 20 mg/ml in destined water (aqua purificata Ph.Eur.).

This stock solution was added to the cultures to obtain the different concentrations of “EFLA 85942” in the following experiments.

The following results were obtained.

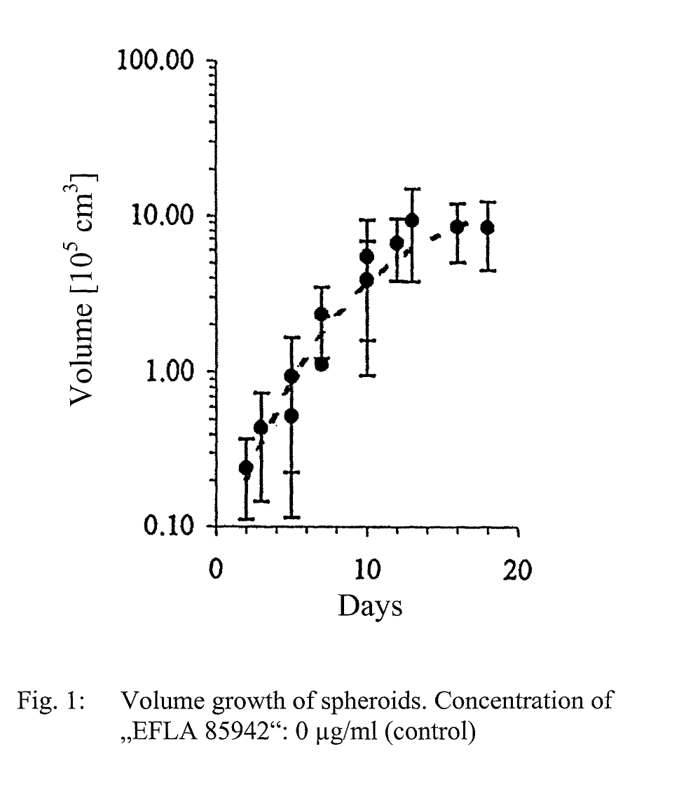

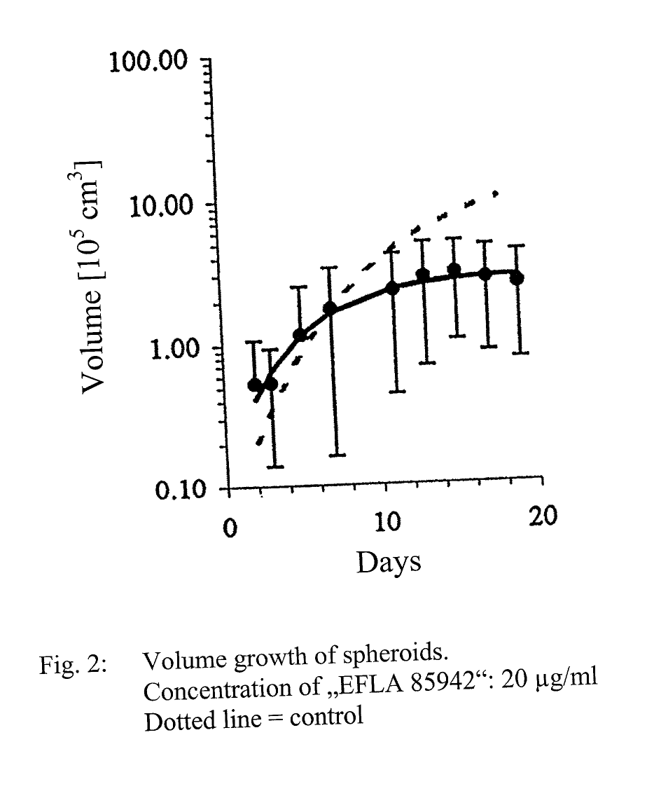

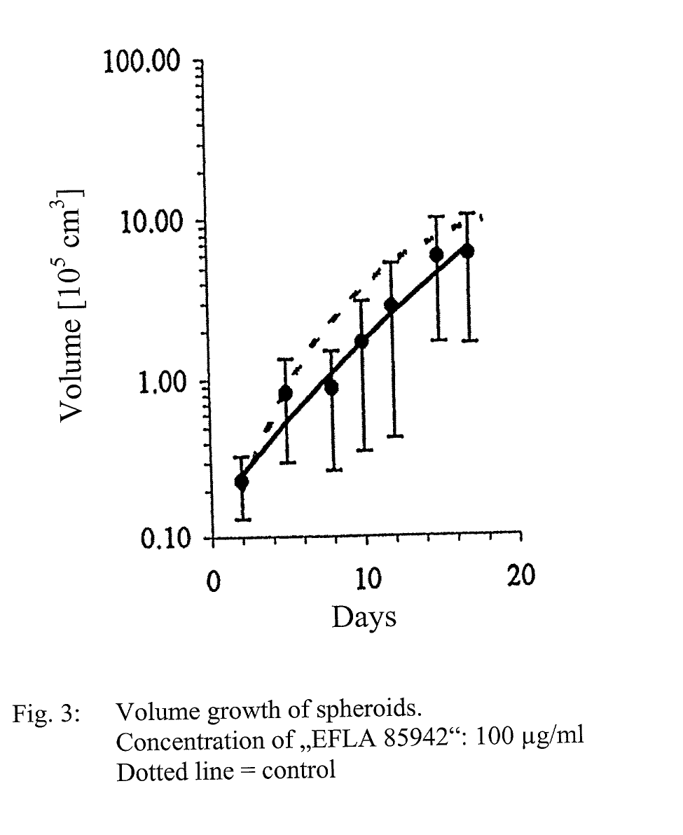

The growth curve of spheroids originating from human colon carcinoma cells under control conditions and after treatment with 20 and 100 μg/ml of “EFLA 85942” are represented in FIGS. 1 to 3.

In all cases the data could be fitted by a Gompertz function which describes a growth kinetic characteristic for the volume increase of malignant tumors invivo. The resulting adaption parameters are listed in table 1.

Both concentrations of “EFLA 85942” cause a systematic and in most areas of the growing period significant decrease of volume growth.

A dependency on concentration after onset of activity (20 μg/ml-100 μg/ml) could not be proven.

Gompertz parameters for description of the volume increase kinetic of multicellular spheroids under the influence of “EFLA 85942”.

TABLE 1

| Parameter | Control | 20 μg/ml | 100 μg/ml |
|---|---|---|---|
| V₀ (10⁵ cm³) | 0.05 | 0.12 | 0.14 |
| A (days⁻¹) | 0.80 | 0.80 | 0.28 |
| B (days⁻¹) | 0.14 | 0.25 | 0.03 |

As expected “EFLA 85942” effected a systematic and in large ranges of the growth length a significant reduction of the volume growth of the spheroids.

But quite surprisingly no concentration dependence could be detected thereby after the beginning of the effect over a broad concentration range.

This effect points to a high therapeutical width.

In addition it was noted quite surprisingly that the with “EFLA 85942” treated spheroides formed no noteworthy necrosis during the whole growth phase.

In control cultures which were not treated with “EFLA 85942” already at diameters of about 200 micrometers were detected central necrosis which increased strongly during the growth phase.

Influence of “EFLA 85942” on the formation of necrosis in spheroids: the thickness of the vital peripheral layer under control conditions and cultivated in the presence of “EFLA 85942” is represented in table 2.

TABLE 2

| | | |
|---|---|---|
| “EFLA 85942” (μg/ml) | 0.0 | 100.0 |
| Thickness of peripheral layer (μm) | 108 +/− 9 | 271 +/− 35 |

The untreated spheroids showed a thickness of the vital peripheral layer of only 108 μm.

In the case of 100 μg/ml, the peripheral layer was drastically thicker than under control conditions.

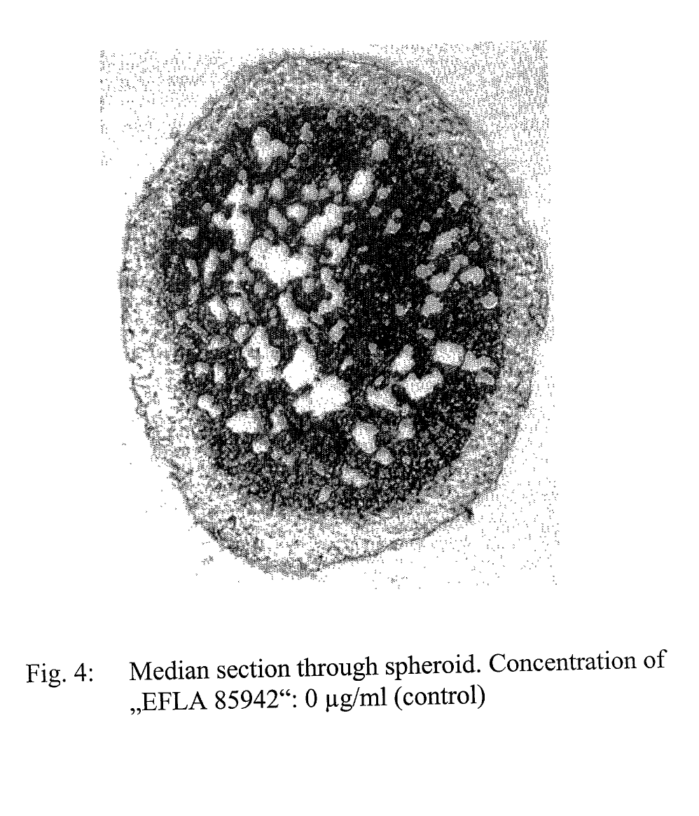

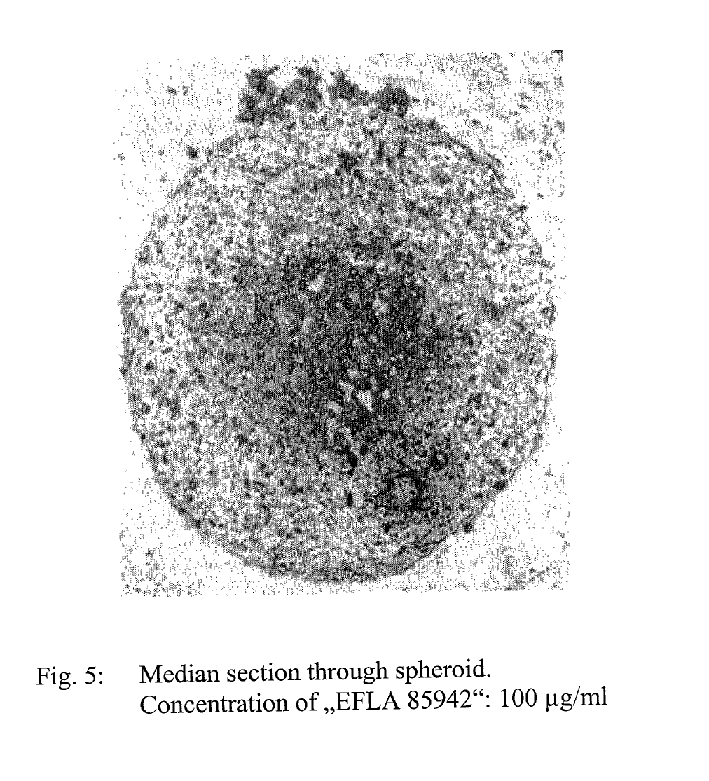

The microscopic pictures in FIGS. 4 and 5 clarify this further. FIGS. 4 and 5 show median sections through spheroids with a diameter of ca. 750 μm.

From this results that untreated spheroids start to form a central necrosis when their diameter is only ca. 220 μm, whereas this process begins not until the diameter has reached ca. 500 μm when treated with 100 μg/ml of Camellia special extract (“EFLA 85942”). The essential result of these experiments is the conclusion that “EFLA 85942” considerably slows down or even suppresses the formation of necrosis.

At the end of the duration of test the untreated spheroids showed only a very thin vital border layer.

This occurance of necrosis is typical for the used experimental model.

This behaviour of the with “EFLA 85942” treated spheroids has not yet been observed.

It is persumed that the known antioxidative effect of the polyphenols can not be responsible alone for this behaviour.

On behalf of this may be used also the results which were obtained from accompanying investigations on singly cells; see example 2.

### EXAMPLE 2

In a first experiment were showed colon carcinoma cells under the influence of “EFLA 85942” in so called adhered culture flasks.

The following concentrations were used:

| | |
|---|---|
| 0,0 μg/ml | (control) |
| 20 μg/ml | of “EFLA 85942” |
| 100 μg/ml | of “EFLA 85942” |
| 200 μg/ml | of “EFLA 85942”. |

The cells were incubated for 72 hours and then examined by microscopy.

Expected was the formation of a so called monolayer-film.

But quite surprisingly was observed the formation of three-dimensional, strongly connected cell aggregates.

Also this behaviour has not yet been observed.

In addition were observed in the culture media nearly no non-adhered single cells.

In a second experiment some few single cells were sowed in non-adhered Petri dishes, with the aim to analyze the influence of “EFLA 85942” onto the colony generation ability of these single cells.

Thereby two variants were carried out.

In experiment A was added “EFLA 85942” to the cells after an initial adhesion on the Petri dish.

TABLE 3
Plating efficiency (PE) values (%) at different
extract concentrations in μg/ml

| | | | | | | | |
|---|---|---|---|---|---|---|---|
| “EFLA 85942” | 0.0 | 0.5 | 1.0 | 5.0 | 10.0 | 15.0 | 20.0 |
| PE | 56 | 61 | 49 | 51 | 53 | 47 | 45 |

In experiment B was added “EFLA 85942” to the cells immediately before the adhesion process.

TABLE 4
Plating efficiency (PE) values (%) as a
function of the extract concentration in μg/ml

| | | | |
|---|---|---|---|
| “EFLA 85942” | 0.0 | 20.0 | 100.0 |
| PE | 52 +/− 4 | 27 +/− 2 | <1 |

In these two experiments could be determined a statistical significant decrease of the colony generation ability which occured in experiment B, in comparison to experiment A, in a drastic stronger extent.

The result of these two experiments, together with the above described formation of three-dimensional cell aggregates, allows the conclusion that on one hand the adhesion with non histo-compatible structures is reduced and that on the other hand the adhesion between single, to the same tissue type belonging cells or cell unions is promoted.

These behaviour characteristics prove that beside the above mentioned antioxidative effect of the polyphenols still further effect mechanisms and/or further active compounds play an important rule.

1. A method for the treatment of human or animal cells, tissue cultures and/or organs outside the human or animal body, wherein said human or animal cells, tissues and/or organs are in need of:
(i) prevention or considerable reduction of the formation of tissue necrosis and/or atrophy and/or the prevention or considerable reduction of the premature mortification of vascularized and non-vascularized cells and cellular tissues/colonies, and
(ii) prevention or considerable reduction of adhesion of a human or animal cell to cells or cell unions of different non-histocompatible tissue types while promoting adhesion of said cell to cells or cell unions of the same tissue type;
comprising administering to said human or animal cells, tissue cultures and/or organs outside the human or animal body and in need of treatment a medicament, a medical care product, a food product or an ingredient for tissue culture media, which comprises a treatment-effective amount of a partial extract of non-fermented Camellia sinensis L., obtained by extracting non-fermented Camellia sinensis L. with a mixture of alcohol and water, wherein said extract comprises phenylchroman derivatives, caffeine, theobromine, glutaminic acid-ethylamide, flavonoides and plant acids.

2. The method according to claim 1, which is for the cultivation and propagation and/or the three-dimensional reconstruction of cartilage cells, gingiva cells, hair root cells, skin cells and skin tissues, retina cells and retina tissue, heart muscle cells and heart muscle tissues, or liver cells and liver tissues.
